(19) United States
(12) Patent Application Publication
(10) Pub. No.: US 2024/0058215 A1
(43) Pub. Date: Feb. 22, 2024

(54) DISPOSABLE FLUID CIRCUITS AND CONTAINERS FOR SUPPRESSING HEMOLYSIS IN STORED RED BLOOD CELLS

(71) Applicant: Fenwal, Inc., Lake Zurich, IL (US)

(72) Inventors: Craig L. Sandford, Buffalo Grove, IL (US); Daniel R. Lynn, Spring Grove, IL (US); Adrienne B. Karpiel, Woodstock, IL (US); Timo Matser, Emmer-Compascuum (NL)

(21) Appl. No.: 18/257,065

(22) PCT Filed: Dec. 17, 2021
(86) PCT No.: PCT/US21/64163

§ 371 Date: Jun. 12, 2023

Related U.S. Application Data
(60) Provisional application No. 63/127,649, filed on Dec. 18, 2020.

(51) Int. Cl. A61J 1/10; A61J 1/14; A61M 1/02

(52) U.S. Cl. CPC A61J 1/10; A61J 1/1468; A61M 1/0218; A61M 2202/0429

(57) ABSTRACT

Medical containers and disposable fluid circuits (kits) including such containers are disclosed. The containers and kit components are made of a plastic composition including polyvinyl chloride and one or more plasticizers such as a citrate ester and an epoxidized vegetable oil are disclosed. Containers made from such compositions are useful in the storage red blood cells. Red blood cell products wherein the red blood cells exhibit a reduced level of hemolysis are also disclosed.

### CROSS-REFERENCE TO RELATED APPLICATIONS

This application claims the benefit of and priority to U.S. Provisional Patent Application Ser. No. 63/127,649, filed Dec. 18, 2020, the contents of which are hereby incorporated by reference.

### FIELD OF THE DISCLOSURE

The present disclosure is directed to medical products such as disposable fluid circuits for the collection and processing of biological fluid including the parts and components that make up the fluid circuit such as, but not limited to, containers for the collection and long-term storage of blood and/or blood components, other containers useful in the processing of biological fluid such as blood and blood components, container ports, blood filters, tubing that defines flow paths in the fluid circuit, and connectors that join two or more tubes of the fluid circuit wherein one or more of the kit components are made from compositions that suppress hemolysis in red blood cells. More particularly, the present disclosure is directed to red blood cell products that exhibit a reduced level of hemolysis after storage for an extended period of time.

### BACKGROUND

Blood components are often separated from whole blood and collected for later transfusion to a patient in need of the separated blood component. For example, platelets may be administered to patients whose ability to make platelets has been compromised due to radiation or chemotherapy. Red blood cells (hereinafter “RBCs”) may be administered to a patient suffering from a loss of blood due to trauma, as a post-chemotherapy treatment, or as part of a treatment of one or more blood borne diseases, such as certain anemias and the like. Unless administered immediately after collection from a donor, some components are typically stored for some period of time prior to transfusion. The storage period may be anywhere from a few days (in the case of platelets) to several weeks (in the case of RBCs).

Prolonged storage of RBCs can (negatively) affect RBC function. In order for e RBCs to be suitable for transfusion to the recipient, RBCs must maintain adequate cell function and metabolism. For example, RBCs must maintain an adequate concentration of adenosine triphosphate (ATP) and 2,3-DPG. In addition, the presence of lactate must not be too high in the stored RBCs. Still further, stored RBCs must have acceptably low levels of hemolysis. Typically, an acceptable level of hemolysis is below 1.0% (in, for example, the U.S.) and 0.8% (in Europe) after 42-day storage.

Hemolysis may vary based on the source of blood as blood from different donors may show different levels of hemolysis. For example, it is possible that within a sample size of 50 donors averaging 0.5% hemolysis, certain donors within the sample size may have RBCs that exceed 1.0% during the storage period.

During storage, concentrated RBCs and the additive solutions in which they are stored are typically kept in a sealed container, usually made of a polymeric material. Most typically, the containers approved for the collection of whole blood and the storage of RBCs are made of a polyvinyl chloride (PVC). Inasmuch as polyvinyl chloride can be somewhat rigid or brittle, a plasticizer is typically incorporated into the PVC. One example of a currently known and used plasticizer for medical grade PVC is di-ethylhexyl phthalate ester or DEHP. Other plasticizers that have been used with PVC or other polyolefin materials include TEH™, and the family of citrate esters described in U.S. Pat. Nos. 4,824,893; 4,710,532 and 4,711,922, the contents of which are incorporated by reference herein. In addition, epoxidized oil is often added as a secondary plasticizer to one or more of the plasticizers described above.

As reported in U.S. Pat. No. 5,026,347 and other literature, such as Rock, et al. “Incorporation of plasticizer into red cells during storage,” Transfusion, 1984; Horowitz et al. “Stablization of RBCs by the Plasticizer, Di(ethylhexyl)phthalate,” Vox Sarguinis, 1985, certain plasticizers may have a beneficial effect on the storage life of RBCs such as hemolysis suppression. As presently understood, plasticizer leached from the sheets that make up the container walls and into the stored blood assists in the suppression of hemolysis.

Perhaps the most widely used plasticizer for PVC blood storage containers is the previously mentioned di-ethyl hexyl phthalate ester or DEHP. DEHP has proven effective as a plasticizer for PVC containers and as a hemolysis suppressant in stored red blood cells during the typical maximum storage period of 42 days. Specifically, DEHP-plasticized PVC containers are effective in maintaining hemolysis levels below the above-mentioned U.S. and EU established criteria and even below 0.5%. The amount of DEHP may be present in the PVC resin across a relatively broad range of concentrations or amounts and still provide acceptable hemolysis levels. (See for example, FIG. 3 which is discussed in greater detail below.)

Despite the beneficial effect of certain leachable plasticizers such as DEHP on hemolysis levels in stored RBCs, the medical community continues to look for alternatives to DEHP. Citrate esters described in U.S. Pat. Nos. 4,824,893; 4,710,532 and 4,711,922, the contents of which have previously been incorporated by reference herein, are effective plasticizers for PVC.

Hemolysis can be visibly detectable in plasma at concentrations as low as 0.5% Many blood collectors judge the quality of the collected blood based upon visual inspection. Accordingly, it would be desirable to provide a medical container made of PVC that has been plasticized with an effective concentration of citrate ester such as BTHC (and optionally an epoxidized oil) wherein average hemolysis is maintained at a level below 0.4% while still providing container properties that make it suitable for the storage of RBCs as well as other blood products and blood components such as whole blood, plasma and platelets.

### SUMMARY

There are several aspects to the subject matter of the present disclosure.

In one aspect, the present disclosure is directed to a disposable fluid circuit for the processing of a biological fluid such as blood or blood components. The fluid circuit includes an access device for withdrawing blood from a blood source, the access device being in openable flow communication with one more container(s) for receiving blood and/or for storing a separated blood component such as red blood cells. The one or more containers include(s) first and second sealed together walls defining and interior chamber for holding red blood cells, wherein at least one of said first and second walls includes a surface facing the interior chamber. The one or more containers and, more particularly, the container walls may be made from a plastic composition including approximately 57-64% wt. polyvinyl chloride and approximately 50-60 phr of n-butyryl-tri-hexyl citrate.

In another aspect, the present disclosure is directed to a container for the storage of red blood cells. The container includes first and second sealed together walls defining and interior chamber for holding red blood cells, wherein at least one of said first and second walls includes a surface facing said interior chamber. The container walls are made of a composition including approximately 57-64% wt. polyvinyl chloride and approximately 50-60 phr of n-butyryl-tri-hexyl citrate.

In a further aspect, the present disclosure is directed to a red blood cell product. The product includes a container having first and second sealed together walls defining and interior chamber for holding red blood cells, wherein at least one of said first and second walls includes a surface facing said interior chamber. The container walls are made of a composition including approximately 57-64% wt. polyvinyl chloride, and approximately 50-60 phr of a citrate ester such as n-butyryl-tri-hexyl citrate.

The red blood cell product further includes red blood cells within said interior chamber wherein said red blood have an average hemolysis level not exceeding 0.4.

The red blood cell product may further include an additive or storage medium.

In a still further aspect, the present disclosure is directed to plasma products and platelet products, respectively. Plasma products include a container having first and second sealed together walls defining and interior chamber for holding thawed plasma, wherein at least one of said first and second walls includes a surface facing said interior chamber. The container walls are made of a composition including approximately 57-64% wt. polyvinyl chloride, and approximately 50-60 phr of a citrate ester such as n-butyryl-tri-hexyl citrate. The thawed plasma within said interior chamber has a Factor VIII activity >70% (70 U/dL; 70 IU/dL: 0.7 U/mL; 0.7 IU/mL); Fibrinogen that is 200 mg/dL and post-storage recovery of IgG and Fibrinogen (mg/dL)≥80%; and a post-storage recovery of von Willibrand factor antigen and Factors II, V, VIII, IX, XI (% activity; U/dL; IU/dL)≥80%.

Platelet products include a container having first and second sealed together walls defining and interior chamber for holding platelets, wherein at least one of said first and second walls includes a surface facing said interior chamber.

The container walls are made of a composition including approximately 57-64% wt. polyvinyl chloride, and approximately 50-60 phr of a citrate ester such as n-butyryl-tri-hexyl citrate. The platelets within said interior chamber have a pH of at least 6.2 at five days of storage.

### BRIEF DESCRIPTION OF THE DRAWINGS

FIG. 1 is a front view of a typical RBC storage container used for storing the red blood cells; and

FIG. 2 is a side view of the container of FIG. 1;

FIG. 3 is a graph comparing hemolysis levels in two different plasticizer systems (DEHP and BTHC) based on test tube studies of blood collected from different donors;

FIG. 4 is a graph showing the relationship between the amount of leached BTHC plasticizer in the stored blood product and the amount of plasticizer in the formulation of the container, expressed as density (lower density indicating a higher level of BTHC in the formulation);

FIG. 5 is a front view of a disposable fluid circuit or blood collection kit including one or more containers made of a composition in accordance with the present disclosure; and

FIG. 6 is a front view of another embodiment of a disposable fluid circuit or blood collection kit including one or more containers made of a composition in accordance with the present disclosure.

### DETAILED DESCRIPTION OF THE EMBODIMENTS

Disclosed herein are disposable fluid circuits such as blood collection kits for the collection and processing of biological fluid including the containers for holding blood and blood components such as, but not limited to, red blood cells (RBCs) and red blood cell products during a period of storage wherein the container is made of a plastic material including at least one extractable agent such as a plasticizer which is capable of suppressing hemolysis in the stored RBC. More particularly, the container typically is made of a polymeric composition such as, but not limited to, polyvinyl chloride and at least one extractable agent that can reduce or suppress hemolysis in RBCs. The RBC product includes concentrated RBCs that have typically been combined with an additive solution selected to maintain cell function and metabolism of the RBCs during prolonged storage (e.g., about 42 days). The red blood cells or RBC product is intended for transfusion to a patient.

As noted above, RBC products typically include RBC concentrate and an additive solution. Concentrated RBCs are derived from whole blood either by manual or automated separation collection techniques which will be known to those skilled in the art. RBC concentrates may include some residual amount of plasma. In one embodiment, the RBC concentrate may have most of its plasma removed as described, for example, in International Application Publication WO/2011/049709, incorporated herein by reference.

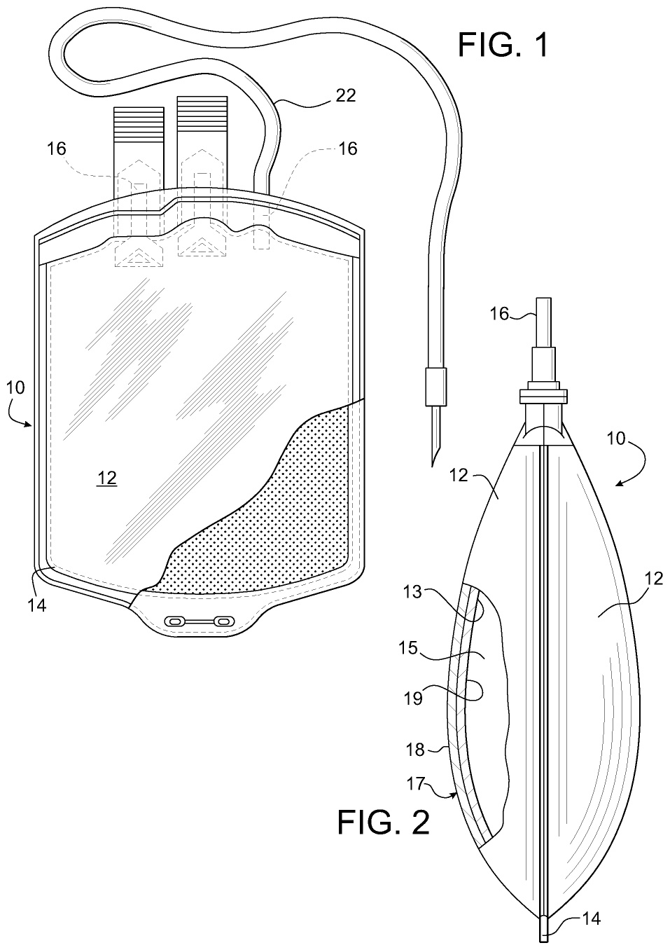

As indicated above, the RBCs and RBC products described herein may be provided in or include a container that is suitable for the long-term storage of RBCs. Preferably, containers for storing the RBC products disclosed herein are made of a polymeric composition. The containers may be permeable to oxygen or at least semi-permeable to oxygen. As shown in FIGS. 1 and 2, container 10 may include one or more container walls 12 which define an interior chamber 15 for receiving the RBCs. In one embodiment, two sheets made of a polymeric material are brought together and sealed along their peripheries 14 by, for example, heat sealing (e.g., RF) to form container 10. Other ways of making container 10 will be known to those of skill in the art and are within the scope of the present disclosure.

As shown in FIG. 2, container wall 12 includes an outer surface 17 and an inner surface 13 which contacts the RBCs stored in the container. In one embodiment, container wall 12 may be made of a multiple sheet (18,19) laminate wherein inner surface 13 is made of one material and outer surface 17 is made of a different material. In one embodiment, the inner surface that is in contact with the blood component within the interior chamber is made of the polymeric composition described herein. Container 10 may include one or more access ports 16 for connection with tubing 22, docking devices and the like to establish flow into and out from the interior chamber 15 of container 10.

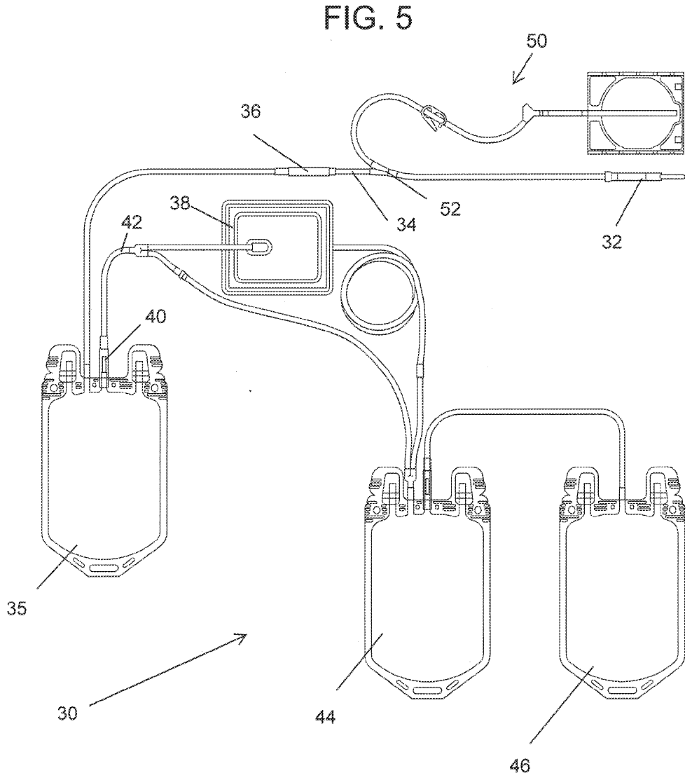

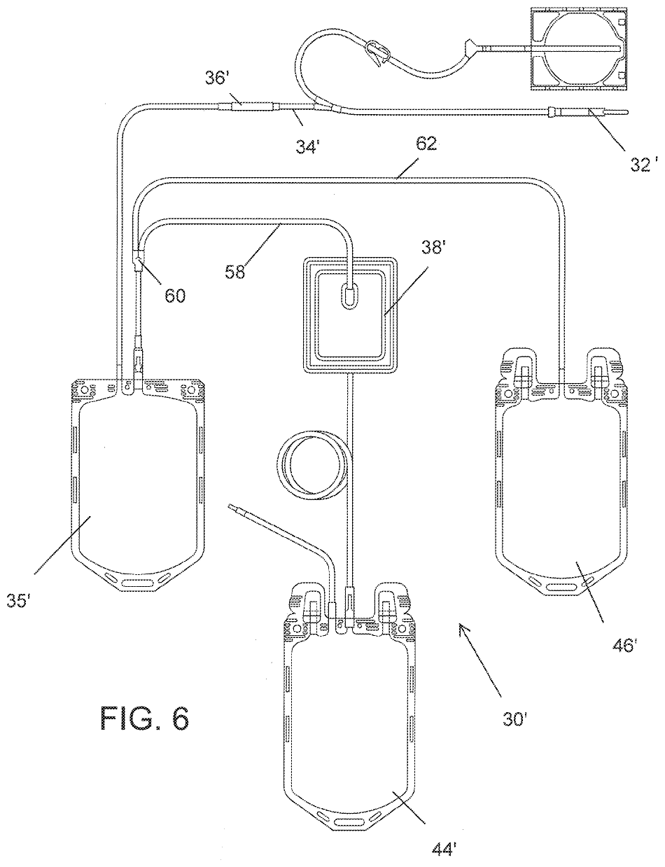

The containers described above may be stand-alone blood or blood component storage containers or may be included as part of a disposable fluid circuit or blood collection kit 30 for collecting and processing blood and blood components as shown in FIGS. 5 and 6 and described below, where in containers are identified by reference numerals 35, 44, 46, 35′, 44′ and 46′, Kit 30 includes a vascular access device such as venipuncture needle 32 for accessing a blood source such as the vascular system of a donor. Venipuncture needle 32 is in openable flow communication with tubing 34 which defines an openable flow path to (whole) blood collection container 35. The flow path of tubing 34 is openable by virtue of a frangible/breakable connector 36 of known type, Thus, whole blood withdrawn from a donor through venipuncture needle 32 and the flow path of tubing 34 is collected in collection container 35 for further processing. Collection container 35 may include an anticoagulant such as CPD (citrate phosphate dextrose) or CPDA1 (citrate phosphate dextrose adenine).

Kit 30 also a includes a leukoreduction filter 38 for removing unwanted white blood cells (leukocytes) from the whole blood. Thus, after collection of whole blood in collection container 35, the whole blood may be expressed from container 35 through outlet port 40 and tubing 42 to downstream leukoreduction filter 38. As the whole blood is passed through filter 38, leukocytes are removed from the whole blood and retained/captured by filter 38. Additional details of leukoreduction filter 38 are set forth in U.S. Pat. No. 9,796,166, which is incorporated by reference. The filtered whole blood is then collected in container 44 and further processed, as necessary. For example, once filtered, the whole blood may be centrifuged (subjected to a hard spin) to separate the plasma from concentrated red blood cells. The separated plasma upper layer may then be expressed to container 46, while leukoreduced red blood cells remain in container 44. Kit 30 may include one or more additional satellite containers such in openable flow communication with container 44 for introducing an additive solution such as Adsol. Kit 30 may also include a pre-donation sampling sub-assembly 50 joined to kit 30 at branch member 52. Pre-donation sampling subassemblies are described in U.S. Pat. No. 6,387,086, also incorporated by reference,

FIG. 6 shows an alternative fluid circuit or blood collection kit 30 ′. In many respects, kit 30′ is identical to kit 30 of FIG. 5. It includes a venipuncture needle 32′ that is in openable flow communication with tubing 34′ which defines an openable flow path to collection container 35′ by virtue of a frangible/breakable connector device 36. As shown in FIG. 6, container 35′ is in openable flow communication with filter 38′ by a flow path defined by tubing 58. Filter 38′ may be configured for filtering a blood component rather than whole blood. For example, filter 38′ may be configured for filtering concentrated red blood cells. In that regard, whole blood collected in collection container 35′ may be subjected to a separation step (such as centrifugation) to separate the plasma component from the red blood cell component. Once separated in container 35′, the lighter plasma component is expressed to container 46′ via branch member 60 and tubing/flow path 62, leaving red cell concentrate in container 35′. An additive solution from a container downstream of filter 38′ may be flowed through filter 38′ to collection container 35′ where it is mixed with the red blood cells. The mixture of red blood cells and additive solution is then filtered through “red cell” filter 38′ and collected in container 44′.

Containers 10, 35, 44, 46, 35′, 44′ and 46′ useful in the collection, processing and/or storage of RBCs as described herein include container walls that are single sheets made in whole or at least in part of a material that includes the polymeric compositions described herein. The single-layered sheets that form wall(s) 12 include an outer surface 17 and an inner surface 13 that contacts the blood component within interior chamber 15. The PVC polymer may be blended with the plasticizer(s) described herein and formed into flat sheets that are sealed together in the manner described above.

By way of example only and not limitation, containers of the type described herein may have a container sheet (wall 12) thickness of between approximately 0.010 to 0.018 inches. They may include a non-smooth or any surface finish that minimizes sheet sticking. For example, for containers that are subjected to steam sterilization (autoclaving), a taffeta finish may be preferred. Typically, containers of the type described herein may have a container volume (i.e., interior chamber volume) of approximately 150 ml to 4 L. The containers of the present disclosure are preferably sterilized by autoclaving and such autoclavable containers may typically have a 70-85 durometer (Shore A).

As noted above, the polymeric composition of the present disclosure is plasticized polyvinyl chloride. Polyvinyl chloride preferably makes up the majority (by wt. %) of the composition as described in greater detail below. The polyvinyl chloride is plasticized with one or more extractable plasticizing agents. In one embodiment, the primary extractable plasticizer may be a citrate ester such as one or more of the citrate esters described in U.S. Pat. Nos. 4,824,893; 4,710,532 and 4,711,922, the contents of which have previously been incorporated by reference. Particularly useful in the composition of the present disclosure is the citrate ester, n-butyryl-tri-hexyl citrate (BTHC).

In addition to the primary extractable plasticizer, the composition of the present disclosure may optionally include a secondary plasticizer selected from the group of epoxidized vegetable oils which may have a lesser effect on RBC hemolysis but may be included to stabilize the composition. Suitable oils for use with the selected citrate ester in the PVC composition include but are not limited to epoxidized soybean oil, epoxidized linseed oil and epoxidized safflower oil.

Compositions made in accordance with the present disclosures may also include minor amounts of one or more additional stabilizers- and, optionally, lubricants and slip agents. Metal stearates, such as zinc stearates or calcium stearates may be included and may be present below 1% and more typically below 0.5%.

In accordance with the present disclosure, the composition includes a concentration of a primary extractable plasticizer(s) such as a citrate ester and, more particularly, n-butyryl-tri-hexyl citrate (BTHC) that is effective in controlling and suppressing hemolysis in stored red blood cells at levels comparable to DEHP-plasticized PVC containers. The concentration of the BTHC primary plasticizer is selected to reduce, suppress and/or control hemolysis for up to about 42 days and maintain average hemolysis levels below 1.0% and, more preferably, below 0.4% at the end of the 42-day storage period when stored with a storage solution such as Adsol. “Average hemolysis” means an average maximum hemolysis based on a minimum sample size of 30 donors, with at least 95% and up to approximately 97% confidence 95% of the time. For sampling sizes greater than 200, the confidence level that average hemolysis is below 0.4% may be above 99.99% (95% of the time).

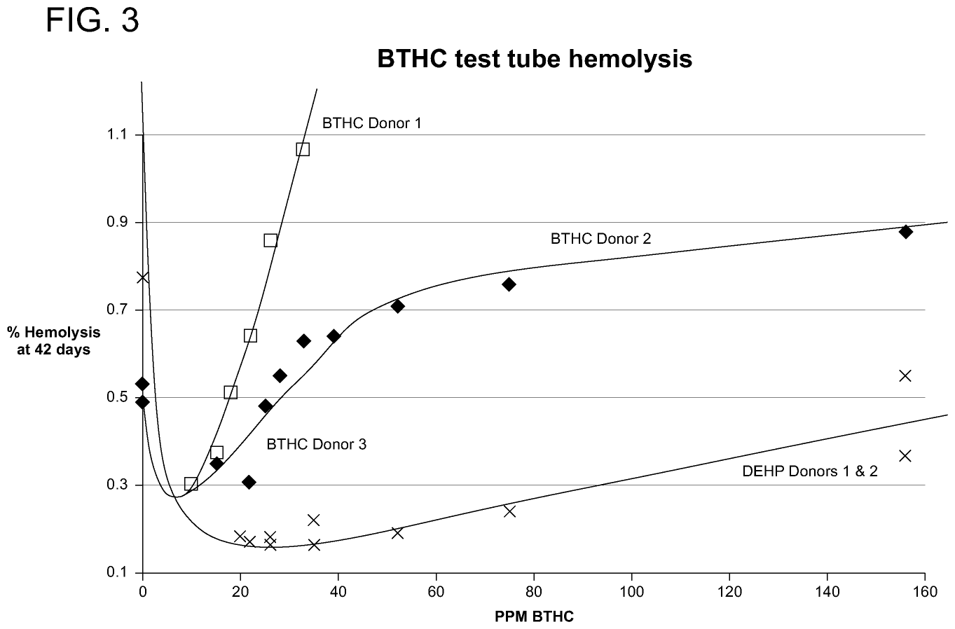

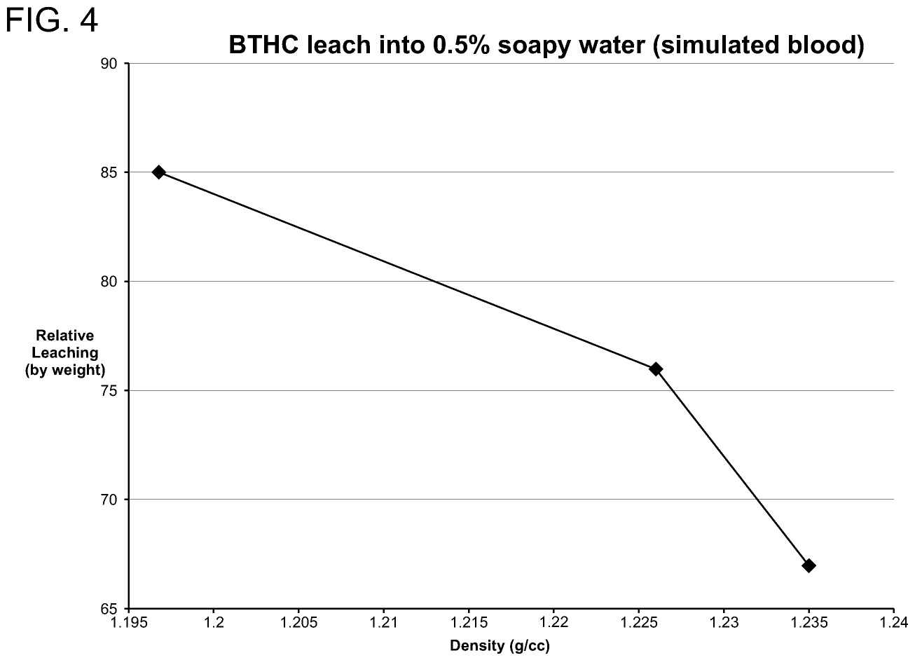

FIG. 3 presents the results of test tube studies wherein red blood cells collected from different donors were combined with varying but fixed amounts (in ppm) of selected plasticizers, in this case DEHP and BTHC. From FIG. 3, it can be seen that DEHP is effective in suppressing and maintaining hemolysis over a relatively broad range of concentrations. During storage in polymeric blood containers or bags, the plasticizer will leach into the stored blood (cells) causing the concentration of plasticizer in the stored red blood cells to increase over the storage period. The amount of plasticizer in the formulation of the plastic container can be predicted from the amount of plasticizer that has leached into the red blood cells. For example, with reference to FIG. 4, it can be seen that the amount of plasticizer that has leached into the blood is correlated to the amount of plasticizer in the formulation of the container (as decreasing density indicates an increasing concentration of BTHC). Thus, returning to FIG. 3, the effects of increasing and decreasing such plasticizer concentrations in the polymeric formulations of the container or bag on % hemolysis of the red blood cells stored therein can be predicted.

As reported in FIG. 3, percent (%) hemolysis levels were recorded at 42 days in Adsol. Per the test tube hemolysis test, samples with DEHP plasticizer showed hemolysis levels well below 0.5% and in some instances below 0.3% over a broad range of concentrations (approximately 15-100 ppm). With respect to % hemolysis levels for blood combined with BTHC plasticizer, acceptable hemolysis levels (below 0.5%) for such samples fell within a much narrower band of concentrations, as also shown in FIG. 3. Inasmuch as test tube hemolysis testing is performed with a constant plasticizer concentration, it will be different than the plasticizer concentration in RBCs in a bag since this concentration will change over time as plasticizer leaches into the RBCs.) With respect to % hemolysis levels for blood combined with BTHC plasticizer, acceptable hemolysis levels (below 0.5%) for such samples fell within a much narrower band of concentrations, as also shown in FIG. 3. Thus, the tube studies described herein and reported in FIG. 3 are indicative of certain hemolysis trends such as (1) there exist optimum concentrations of plasticizer for producing low hemolysis results (2) the optimum concentration range for BTHC is lower and much narrower than the optimum range for DEHP (3) BTHC concentrations above (and below) the optimum range are less effective in maintaining or suppressing hemolysis. Test tube hemolysis tests are described in U.S. Patent Application Publication No. US 2021/0205173 which is incorporated herein by reference. Thus, in one embodiment, the composition of the present disclosure includes approximately 57%-64% by weight (% wt.) polyvinyl chloride and approximately 50-60 phr of a primary plasticizer such as the previously mentioned n-butyryl-tri-hexyl citrate (BTHC). Stated as a percentage of the entire composition, the wt. % of primary plasticizer (e.g., BTHC) is approximately 29-36 wt, %, as determined by an extraction assay. (The extraction assay was carried out by first dissolving 50 mg pf a PVC-BTHC foil in tetrahydrofuran (THF). After dissolution, 10.0 ml of methanol was added and the mixture was shaken in a vortex mixer. After the precipitate had subsided, the upper clear solution was injected with an injection volume of 50 μL at a flow rate of 1.50 ml/min into the chromatographic system including a Inertsil ODS-2 4.6 250 MM, Spin column available from GL Sciences). As shown in the Tables below, amounts of n-butyryl-tri-hexyl citrate outside the ranges described above may not be as effective in suppressing hemolysis during storage of RBCs in Adsol for 42 days. By way of background, other hemolysis-suppressing plasticizers, such as DEHP, which are commonly included in PVC formulations may be present in concentrations or amounts over a much broader range. In accordance with the present disclosure, however, the ratio of n-butyryl-tri-hexyl citrate to PVC resin represents a narrower range for the hemolysis-suppressing plasticizer BTHC than the range for DEHP in PVC-DEHP formulations. As a result, in commercial applications for the storage of RBCs there has been no need to precisely control DEHP levels in the DEHP plasticized RBC container.

In a more particular embodiment, the composition of the present disclosure may include approximately 57%-64% wt. polyvinyl chloride and a total plasticizer content of approximately 39%-43% wt. wherein the plasticizer(s) include(s) a primary plasticizer such as n-butyryl-tri-hexyl citrate (BTHC) and a secondary plasticizer such as an epoxidized oil. In this more particular embodiment, the epoxidized oil is selected from the group of epoxidized soybean oil and epoxidized linseed oil. As a percentage of the entire composition, BTHC may make up approximately 29%-36% wt. and the epoxidized vegetable oil may make up 3%-11% wt. The amount of epoxidized oil may be adjusted within the above-stated range or even slightly beyond such range provided that the percentage or amount (phi) of the primary n-butyryl-tri-hexyl citrate (BTHC) plasticizer remains within the ranges disclosed herein. Thus, for example, a ratio of epoxidized oil to citrate ester (such as BTHC) may typically be between approximately 1:3 and 1:10 and more preferably 1:4 or 1:5. As used herein, the term “approximately” includes a difference of ±0.5%-1.0%. More particular examples of compositions in accordance with the present disclosure (expressed as phr of BTHC/epoxidized vegetable oil and as a percentage by weight of BTHC in the polymeric composition) are set forth in Table 1 below.

TABLE 1

| | Approximate phr (parts per | | |
|---|---|---|---|
| | hundred resin) | | |
| Sample | BTHC | Epoxidized Oil | % BTHC |
| 1 | 60 | 15 | 34.3 |
| 2 | 58.4 | 14.6 | 33.8 |
| 3 | 56.8 | 14.2 | 33.2 |
| 4 | 56 | 14 | 32.9 |
| 5 | 55.2 | 13.8 | 32.7 |
| 6 | 53.6 | 13.4 | 32.1 |
| 7 | 52 | 13 | 31.5 |

As noted above, red blood cell products (which include the concentrated RBCs and may further include a synthetic storage media) stored in containers made from compositions of the type described herein exhibit a hemolysis level of below 0.4% at 42 days of storage. The synthetic storage medium may be, but is not limited to, AS-1 (Adsol), a commercially available storage solution.

Hemolysis levels for red blood cell products stored in Adsol (and one product stored in SAGM*) for up 42 days in containers made from PVC plasticized with different amounts of BTHC are reported below. As shown in Table 2, percentages of BTHC within the range of 29%-36% resulted in low average hemolysis levels (i.e., below 0.4%). Using a 32.8% BTHC as a representative percentage of BTHC, % BTHC below such representative percentage still resulted in low average hemolysis levels as did % BTHC slightly above such representative percentage and up to about 36% BTHC. On the other hand, formulations with percentages of BTHC above 36% resulted in higher hemolysis levels (i.e., above 0.4% at 42 days of storage in Adsol). Red blood cell products stored in Adsol and containers made of formulations described herein may have a mean % hemolysis at Day 42 of approximately 0.319.

TABLE 2

| | | Hemolysis (day 42) | |
|---|---|---|---|
| Formulation | % BTHC | in Adsol | # of units |
| 1 | 31.6 | 0.29 | 5 |
| 2 | 31.9 | 0.32 | 5 |
| 3 | 32.6 | 0.33 | 5 |
| 4 | 32.8 | 0.33 | 10 |
| 5 | 32.8 | 0.31 | 10** |
| 6 | 33.1 | 0.30 | 5 |
| 7 | 33.3 | 0.34 | 10 |
| 8 | 33.9 | 0.33 | 5 |
| 9 | 34.5 | 0.34 | 10 |
| 10 | 35.1 | 0.33 | 5 |
| 11 | 34.5 | 0.31 | 10** |
| 12 | 36.4 to | 0.45 | 10** |
| | 38.2 | | |

**Pooled and split units

When one compares the formulations 5 and 12 (part of a paired study), it will be appreciated that % BTHC above the range described herein i.e., greater than 36 wt % results in greater average hemolysis than in formulations where the wt % of BTHC is below 36%. Additional data for the paired study is also included below in Table 3.

TABLE 3

| % BTHC in PVC | 36.4 to 38.2 | 32.8 |
|---|---|---|
| Mean | 0.45 | 0.31 |
| Median | 0.43 | 0.3 |
| Std. Dev. | 0.18 | 0.31 |
| Min | 0.24 | 0.19 |
| Max | 0.87 | 0.63 |
| N | 10 | 10 |
| T-Test | 0.00014 | |
| % diff | 45.20% | |

As noted above, polyvinyl chloride makes up most of the polymeric composition. For example, per 100 parts of the polymeric resin (PVC), the total plasticizer content may be 72±5 of which approximately 57 phr may be BTHC and approximately 15 phr may be the epoxidized vegetable (soybean) oil (ESO). Examples of other suitable formulations based on plasticizer (and more particularly BTHC) content are set forth in Table 3.

TABLE 4

| | Total and individual plasticizer content (phr) | | | | | | | | | |
|---|---|---|---|---|---|---|---|---|---|---|
| Formulation | 1 | 2 | 3 | 4 | 5 | 6 | 7 | 8 | 9 | 10 |
| Total | 67 | 68 | 69 | 70 | 71 | 72 | 73 | 74 | 75 | 76 |
| Plasticizer | | | | | | | | | | |
| BTHC | 53.1 | 53.9 | 54.6 | 55.4 | 56.2 | 57 | 57.8 | 58.6 | 59.4 | 60.2 |
| ESO | 13.9 | 14.1 | 14.4 | 14.6 | 14.8 | 15 | 15.2 | 15.4 | 15.6 | 15.8 |

Containers made from the compositions described herein may also be suitable for storing blood components other than RBCs such as plasma and/or platelets or for storing whole blood. Containers in accordance with the present disclosure are suitable for the storage of plasma in that that plasma that has been thawed has a Factor VIII activity >70% (70 U/DI; 70 IU/DI; 0.7 U/MI; 0.7 IU/MI); Fibrinogen ≥200 mg/DI and post-storage recovery of IgG and Fibrinogen (mg/DI) ≥80%; and a post-storage recovery of von Willibrand factor antigen and Factors II, V, VIII, IX, XI (% activity; U/DI; IU/DI) ≥80%.

Platelets stored in containers as described herein maintained a Ph level of ≥6.2 on Day 5 of storage at least 95% of the time with 95% confidence and had a mean platelet product Ph (22° C.) at Day 5 of approximately 7.3281.

Finally, in addition to being well suited for manufacturing containers, and more particularly the container walls, used to collect or store red blood cells, plasma and/or platelets, the compositions described herein may also be suited for manufacturing other component parts of the disposable fluid circuit (kits 30 and 30′) such as the tubing ports associated with the containers, the tubing defining the flow paths of the circuit, the filter housings and ports, the walls of the sampling pouch 51, 51′, and the branched members/connectors and the frangible connectors described above.

While the kits, containers, products and compositions disclosed herein have been described in connection with various embodiments, it will be apparent to those skilled in the art that modifications and variations may be made thereto without departing from the spirit and scope of the invention.

### Other Examples

Aspect 1. A disposable fluid circuit for processing a biological fluid comprising: an access device for withdrawing blood from a blood source, said access device being in openable flow communication with one or more containers for receiving blood or a blood component, said one or more containers comprising first and second sealed together walls defining and interior chamber for holding blood or a blood component, wherein at least one of said first and second walls includes a surface facing said interior chamber comprising: polymeric composition comprising approximately 57-64% wt. of polyvinyl chloride; approximately 50-60 phr of a citrate ester consisting essentially of n-butyryl-tri-hexyl citrate.

Aspect 2. The disposable fluid processing set of Aspect 1 further comprising a leukoreduction filter located between a container for receiving whole blood from said blood source and a second container.

Aspect 3, The disposable fluid circuit of Aspect 1 wherein said composition further comprises an epoxidized oil.

Aspect 4. The disposable fluid circuit of Aspect 3 wherein said epoxidized oil comprises epoxidized soybean oil.

Aspect 5. The disposable fluid circuit of claim 3 wherein said epoxidized oil comprises epoxidized linseed oil.

Aspect 6, The disposable fluid circuit of any one of Aspects 1 through 5 wherein said extractable plasticizer consists essentially of 29%-36% wt, of said n-butyryl-tri-hexyl citrate.

Aspect 7. The disposable fluid circuit of any one of Aspects 1 through 6 further comprising one or more stabilizers, co-stabilizers and slip agents.

Aspect 8. The disposable fluid circuit of any one of Aspects 1 through 7 wherein the ratio of epoxidized oil to n-butyryl-tri-hexyl citrate is between 1:3 and 1:10.

Aspect 9. The disposable fluid circuit of any one of Aspects 2 through 8 further comprising a third container in openable flow communication with said second container.

Aspect 10. The disposable fluid circuit of Aspect 9 wherein each of said container for receiving whole blood, second container and third container comprises first and second sealed together walls defining and interior chamber for holding blood or a blood component, wherein at least one of said first and second walls includes a surface facing said interior chamber comprising: polymeric composition comprising approximately 57-64% wt. of polyvinyl chloride; approximately 50-60 phr of a citrate ester consisting essentially of n-butyryl-tri-hexyl citrate.

Aspect 11. A container for the storage of red blood cell comprising first and second sealed together walls defining and interior chamber for holding red blood cells, wherein at least one of said first and second walls includes a surface facing said interior chamber comprising a composition comprising: a resin comprising approximately 57-64% wt. of polyvinyl chloride; approximately 50-60 phr of a citrate ester consisting essentially of n-butyryl-tri-hexyl citrate.

Aspect 12. The container of Aspect 11 further comprising an epoxidized oil.

Aspect 13. The container of Aspect 12 wherein said epoxidized oil comprises epoxidized soybean oil.

Aspect 14. The container of Aspect 12 wherein said epoxidized oil comprises epoxidized linseed oil.

Aspect 15. The container of any one of Aspects 11 through 14 consisting essentially of 29%-36% wt. of said n-butyryl-tri-hexyl citrate.

Aspect 16. The container of any one of Aspects 11 through 15 wherein said composition further comprises one or more stabilizers, co-stabilizers and slip agents.

Aspect 17. A red blood cell product comprising: a container comprising first and second sealed together walls defining and interior chamber for holding red blood cells, wherein at least one of said first and second walls includes a surface facing said interior chamber, said container walls comprising a composition including: approximately 57-64% wt. of polyvinyl chloride; approximately 50-60 phi of a citrate ester consisting essentially of n-butyryl-tri-hexyl citrate; red blood cells within said interior chamber wherein said red blood have an average hemolysis level below 0.4 at 42 days of storage.

Aspect 18. The red blood cell product of Aspect 17 further comprising a synthetic storage medium.

Aspect 19. The red blood cell product of any one of Aspects 17 or 18 wherein the amount of said citrate ester in said composition is no greater than 36% wt.

Aspect 20. The red blood cell product of any one of Aspects 14 through 19 wherein said composition further comprises epoxidized oil is selected from the group consisting of epoxidized soybean oil and epoxidized linseed oil.

Aspect 21. A platelet product comprising: a container comprising first and second sealed together walls defining and interior chamber for holding platelets, wherein at least one of said first and second walls includes a surface facing said interior chamber, said container walls comprising a composition including: approximately 57-64% wt. of polyvinyl chloride; approximately 50-60 phr of a citrate ester consisting essentially of n-butyryl-tri-hexyl citrate; platelets within said interior chamber wherein said platelets have a pH of at least 6.2 at five days of storage.

Aspect 22. A plasma product comprising: a container comprising first and second sealed together walls defining and interior chamber for plasma that can be frozen followed by thawing, wherein at least one of said first and second walls includes a surface facing said interior chamber, said container walls comprising a composition including: approximately 57-64% wt. of polyvinyl chloride; approximately 50-60 phr of a citrate ester consisting essentially of n-butyryl-tri-hexyl citrate; thawed plasma within said interior chamber wherein said plasma has a Factor VIII activity >70% (70 U/dL; 70 IU/dL; 0.7 U/mL; 0.7 IU/mL); Fibrinogen that is ≥200 mg/dL and post-storage recovery of IgG and Fibrinogen (mg/dL) ≥80%; and a post-storage recovery of von Willibrand factor antigen and Factors II, V, VIII, IX, XI (% activity; U/dL; IU/dL) ≥80%.

1. A disposable fluid circuit for processing a biological fluid comprising:
a. an access device for withdrawing blood from a blood source, said access device being in openable flow communication with one or more containers for receiving blood or a blood component, said one or more containers comprising first and second sealed together walls defining an interior chamber for holding blood or a blood component, wherein at least one of said first and second walls includes a surface facing said interior chamber comprising:
i. a polymeric composition comprising approximately 57-64% wt. of polyvinyl chloride; and
ii. approximately 50-60 phr of a citrate ester consisting essentially of n-butyryl-tri-hexyl citrate.

2. The disposable fluid circuit of claim 1 further comprising a leukoreduction filter located between a container for receiving whole blood from said blood source and a second container.

3. The disposable fluid circuit of claim 1 wherein said composition further comprises an epoxidized oil.

4. The disposable fluid circuit of claim 3 wherein said epoxidized oil comprises epoxidized soybean oil.

5. The disposable fluid circuit of claim 3 wherein said epoxidized oil comprises epoxidized linseed oil.

6. The disposable fluid circuit of claim 1 wherein said polymeric composition consists essentially of 29%-36% wt. of said n-butyryl-tri-hexyl citrate.

7. The disposable fluid circuit of claim 1 further comprising one or more stabilizers, co-stabilizers and slip agents.

8. The disposable fluid circuit of claim 1 wherein the ratio of epoxidized oil to n-butyryl-tri-hexyl citrate is between 1:3 and 1:10.

9. The disposable fluid circuit of claim 2 further comprising a third container in openable flow communication with said second container.

10. The disposable fluid circuit of claim 9 wherein each of said container for receiving whole blood, second container and third container comprises first and second sealed together walls defining an interior chamber for holding blood or a blood component, wherein at least one of said first and second walls includes a surface facing said interior chamber comprising:
a. a polymeric composition comprising approximately 57-64% wt. of polyvinyl chloride; and
b. approximately 50-60 phr of a citrate ester consisting essentially of n-butyryl-tri-hexyl citrate.

11. A container for the storage of red blood cell comprising first and second sealed together walls defining an interior chamber for holding red blood cells, wherein at least one of said first and second walls includes a surface facing said interior chamber comprising a composition comprising:
a. a resin comprising approximately 57-64% wt. of polyvinyl chloride; and
b. approximately 50-60 phr of a citrate ester consisting essentially of n-butyryl-tri-hexyl citrate.

12. The container of claim 11 further comprising an epoxidized oil.

13. The container of claim 12 wherein said epoxidized oil comprises epoxidized soybean oil.

14. The container of claim 12 wherein said epoxidized oil comprises epoxidized linseed oil.

15. The container of claim 11 consisting essentially of 29%-36% wt. of said n-butyryl-tri-hexyl citrate.

16. The container of claim 11 wherein said composition further comprises one or more stabilizers, co-stabilizers and slip agents.

17. A red blood cell product comprising:
a. a container comprising first and second sealed together walls defining an interior chamber for holding red blood cells, wherein at least one of said first and second walls includes a surface facing said interior chamber, said container walls comprising a composition including:
i. approximately 57-64% wt. of polyvinyl chloride; and
ii. approximately 50-60 phr of a citrate ester consisting essentially of n-butyryl-tri-hexyl citrate; and
b. red blood cells within said interior chamber wherein said red blood have an average hemolysis level below 0.4 at 42 days of storage.

18. The red blood cell product of claim 17 further comprising a synthetic storage medium.

19. The red blood cell product of claim 17 wherein the amount of said citrate ester in said composition is no greater than 36% wt.

20. The red blood cell product of claim 17 wherein said composition further comprises epoxidized oil, wherein the epoxidized oil is selected from the group consisting of epoxidized soybean oil and epoxidized linseed oil.

21. A platelet product comprising:
a. a container comprising first and second sealed together walls defining an interior chamber for holding platelets, wherein at least one of said first and second walls includes a surface facing said interior chamber, said container walls comprising a composition including:
b. approximately 57-64% wt. of polyvinyl chloride;
c. approximately 50-60 phr of a citrate ester consisting essentially of n-butyryl-tri-hexyl citrate; and
d. platelets within said interior chamber wherein said platelets have a pH of at least 6.2 at five days of storage.

22. A plasma product comprising:
a. a container comprising first and second sealed together walls defining an interior chamber for plasma that can be frozen followed by thawing, wherein at least one of said first and second walls includes a surface facing said interior chamber, said container walls comprising a composition including:
b. approximately 57-64% wt. of polyvinyl chloride;
c. approximately 50-60 phr of a citrate ester consisting essentially of n-butyryl-tri-hexyl citrate; and
d. thawed plasma within said interior chamber wherein said plasma has a Factor VIII activity >70% (70 U/dL; 70 IU/dL; 0.7 U/mL; 0.7 IU/mL); Fibrinogen that is 200 mg/dL and post-storage recovery of IgG and Fibrinogen (mg/dL) ≥80%; and a post-storage recovery of von Willibrand factor antigen and Factors II, V, VIII, IX, XI (% activity; U/dL; IU/dL)≥80%.
